(19) United States
(12) Patent Application Publication
(10) Pub. No.: US 2023/0047344 A1
(43) Pub. Date: Feb. 16, 2023

(54) ELECTRONIC ADD-ON MODULE FOR INJECTION DEVICES

(71) Applicant: Ypsomed AG, Burgdorf (CH)

(72) Inventors: Simon Scheurer, Bern (CH); Christian Schrul, Burgdorf (CH); Mario Bernhard, Burgdorf (CH); Lukas Hans Frey, Langenthal (CH)

(21) Appl. No.: 17/976,310

(22) Filed: Oct. 28, 2022

Related U.S. Application Data
(63) Continuation of application No. PCT/EP2021/059606, filed on Apr. 14, 2021.

(30) Foreign Application Priority Data
Apr. 30, 2020 (CH) 00509/20

(51) Int. Cl. A61M 5/24; A61M 5/31; A61M 5/315

(52) U.S. Cl. CPC A61M 5/2422; A61M 5/3135; A61M 5/3158; A61M 2205/60

(57) ABSTRACT

An electronic add-on module for an injection device includes a longitudinal axis and a discharge button arranged at a proximal end and configured to be movable in a distal direction along the longitudinal axis for discharging a liquid medication out of a container of the injection device. The add-on module includes a sensor unit, a receiving region adapted to the shape of the housing of the injection device for placing on along the longitudinal axis thereof, and a holding mechanism configured to releasably secure the add-on module against moving axially relative to the injection device. The receiving region is configured such that the discharge button can be accessed or contacted when the add-on module is on the injection device. The add-on module comprises a release element configured to be moved solely on a plane perpendicular to the longitudinal axis in a release movement for releasing the holding mechanism.

### CROSS REFERENCE TO RELATED APPLICATIONS

This application claims priority to International Patent Application No. PCT/EP2021/059606, filed Apr. 14, 2021, entitled “ELECTRONIC ADD-ON MODULE FOR INJECTION DEVICES,” which in turn claims priority to Swiss Patent Application No. 00509/20, filed Apr. 30, 2020, entitled “ELECTRONIC ADD-ON MODULE FOR INJECTION DEVICES”, each of which is incorporated by reference herein, in the entirety and for all purposes.

### TECHNICAL FIELD

The present disclosure relates to the field of medical injection devices for administering liquid substances including medications or medical substances such as insulin and hormone preparations, and provides portable electronic add-on modules configured to be mounted on such medical injection devices.

### BACKGROUND

Different infusion and injection devices can deliver to patients a controlled, subcutaneous, or intramuscular administration of a dose of a medication from a container. While, in infusion systems, a cannula remains in the patient's body during a long period of time, injection devices are removed from the injection site after each delivery. In disposable injection devices, the container, such as a pre-filled syringe, is not intended to be replaced or refilled by the patient. However, in reusable injection devices, the container, such as a carpule, can be refilled or replaced by the patient. An automatic injection device has a motor or a tensioned spring as an energy source for driving a piston rod and for moving a piston in the reservoir, while, when discharging with a manual injection device, the piston rod is moved directly by a force of the patient.

Diabetes is often treated by delivering insulin by means of an injection device in the form of an insulin-pen with an adjustable dose selection. The pen has an elongated housing with a distal end for receiving a cannula or hollow needle and a proximal end facing away from the injection site. An insulin pen can be operated automatically or manually as a disposable or reusable pen. The insulin dose to be delivered is normally set by turning a dosing button and, at the same time, controlling a dose display of the insulin pen. In order to monitor insulin delivery in real time, e.g., in order to prevent incorrect use or to track several deliveries over a longer period, information relating to insulin type, dose size, and time, as well as further circumstances of each dose delivery, can be collected.

For this purpose, EP 3572107 A1 discloses an electronic add-on module, which can be mounted laterally on an injection device and fastened or clamped thereto by means of a snap connection. A snap mechanism comprises two elastic wings on the add-on module which define a receiving area for the injection device. The add-on module comprises a sensor for detecting a state of the injection device and/or for monitoring an ongoing discharge operation.

WO 2007/107564 A1 describes an add-on module for an injection device with a dose setting button arranged on the proximal end of the injection device. Before or after use of the injection device, a distal end of the injection device is protected by a device cap which engages in a coupling element of the injection device and is thereby protected against unintentional release in the distal direction. After removal of the device cap, the add-on module can also engage in the coupling element and thereby secure the module against a proximal movement. The add-on module, in turn, has its own coupling elements, which allow an adapted device cap to be fastened thereto when the add-on module is mounted.

WO 2018/036938 A1 discloses an add-on module that engages with a pin, which is mounted in a pretensioned state, into a recess in the housing of an autoinjector and thereby secures the add-on module against axial displacement. This engagement is released by rotating a button or lever which projects beyond a proximal end face of the add-on module and is guided by two fingers of the user, whereupon the add-on module can be removed again. The autoinjector comprises a trigger button which is completely surrounded or covered by the fitted add-on module and at best, the trigger button can be operated indirectly by a user.

WO 2003/011373 A1 describes a modular injection device with a reusable dosing and actuation module comprising a counting and display device and having a disposable reservoir module comprising a mechanism holder and a carpule holder. A locking engagement by a locking element on a locking ring of the dosing and actuation module in a notch of the mechanism holder is released by actuating an unlocking button on the locking ring and counter to a return spring in the radial direction. A release lock allows for releasing the locking engagement solely at the end of the discharge movement when a proximal actuation element is moved maximally in the distal direction. In order to completely insert and lock the reservoir module, it is necessary to simultaneously press both the actuation element against a spring, which is axially supported on the housing of the dosing and actuation module, and the unlocking button.

### SUMMARY

Disclosed are safe injection systems that include an injection device and an add-on module releasably fastened thereto in which a user may clearly distinguish between an actuation of a discharge mechanism for injecting a dose and an actuation of a release mechanism for releasing the add-on module from the injection device.

In a first aspect, an electronic add-on module may be releasably mounted or pushed onto an injection device along a longitudinal axis of the device, which connects at a distal end on the injection side to an opposite proximal end of the injection device. The injection device may include a device housing, which may contain or surround a delivery mechanism for parenteral subcutaneous or intramuscular delivery of medications from a container of the injection device. The injection device may include a discharge button at its proximal end, which may be moved by a user in the distal direction along a longitudinal axis of the injection device and may thereby cause or trigger a discharge. The discharge button may be directly touched and operated by the user even when the add-on module is mounted, and may not be surrounded or covered by the add-on module. The add-on module may include a sensor unit for detecting processes or states of the injection device, and a processor unit for evaluating and/or processing signals of the sensor unit. The add-on module may further include an energy store for supplying energy to the sensor unit and/or the processor unit and a communications unit for wireless communication of signals from the sensor unit and/or of data of the processor unit.

A holding mechanism of the add-on module may secure at least the sensor unit of the mounted add-on module against axial movement relative to the injection device. The add-on module may include a release element or release control device, which may be operated by a user in a release movement for releasing the holding mechanism and for releasing the add-on module from the injection device. The release movement of the release element may involve a movement solely in a plane perpendicular to the longitudinal axis of the injection device or a linear movement in a direction at least approximately perpendicular to the longitudinal axis. The release movement may be a purely rotational movement about the longitudinal axis or a purely linear movement in a direction primarily perpendicular to the longitudinal axis, e.g., with a minimum deviation of at most 25° or at most 10° to being axially perpendicular, or completely without an axial movement component. The release movement may not involve a screwing movement in the longitudinal direction and the release movement may not involve tilting of a lever about an axis different from the longitudinal axis. The release movement may be the only action to be taken by the user; and for instance no additional releasing at the add-on module and no specific state of the injection device are required. Due to the release movement of the release element being at least approximately perpendicular to the activation direction of the discharge button, the risk of the two movements being confused may be minimized compared to an injection system in which both a discharge button and a release element are pressed or pushed in the same direction.

The injection device itself may not be configured to communicate with a further device, but may be adapted to interact with the add-on module, e.g., by suitable openings in the device housing or by movable actuators such as magnets or sliding contacts, the positions of which may be clearly detected by the sensor of the add-on module. The injection device may be a disposable injection device, e.g., without the possibility of container replacement by a user, or may be a reusable injection device. The injection device may further be an injection device with variable dose setting, comprising a dose setting button arranged at the proximal end for adjusting a dose to be discharged by means of a rotational and/or screwing movement. The dose setting button may also be directly gripped and operated by the user when the add-on module is mounted, and may not be surrounded or otherwise covered by the add-on module. The injection device may also be a single-use device, e.g., provided for one-time delivery of a fixed dose. The injection device may either be operated manually by transferring a discharge force of the user to the discharge button, or may include a pretensioned spring releasable for an automatic discharge by actuating the discharge button.

The add-on module may generally be configured in the shape of a sleeve and may include a rigid receiving region with an opening adapted to the cross-section of the injection device for receiving the injection device, so that, when mounted, the injection device may not be removed from the receiving region transversely to the longitudinal axis or even fall out of the receiving region. Without adversely affecting the mutual alignment of the add-on module and injection device, the receiving region may include a lateral opening or a transparent window through which a label on the device housing is visible. An axial length of the add-on module may be selected such that a dose display window of the injection device for displaying a set dose is not covered by the mounted add-on module, and the dosage display may remain directly visible for the user. Accordingly, the sensor unit may not be configured to read a displayed dose, but may be detected based on an optical, mechanical, or electrical—including magnetic or inductive—operations or states in the injection device. The sensor unit may include at least one, single-axis or multi-axis acceleration sensor, a single-axis or multi-axis gyroscope sensor, an inertial sensor, a piezo-based, structure-borne sound microphone, or a comparable sensor for detecting movements of the injection device. These movements may include both movements of the injection device in space, which may be carried out by a user and by movements of the injection device which are caused by a mechanism in the injection device—for example, oscillations, vibrations, or sound waves of a discharge click or other device operations. For this purpose, in the mounted state, a lateral face of the receiving region of the add-on module may rest as tightly as possible against a bearing face of the injection device.

In embodiments, the release movement may be a linear movement carried out by the user by pushing or sliding the release element. In contrast to a rotational movement, this linear movement may require an alignment of the add-on module to the user, which may constitute a noticeable difference from a rotationally-symmetrical discharge button and make it more difficult for the user to confuse these movements.

In embodiments, the release element may be provided on the distal half of the add-on module, or at the distal end of the add-on module. As a result, the release element may be maximally distanced from the discharge button, which may make confusion of the aforementioned movements even more difficult.

In embodiments, the release element may be a component of a handle for one-handed gripping and guiding of the add-on module and the inserted injection device. The handle may surround the receiving region and may include a length of at least half the hand width of a user or of at least 3 cm in the direction of the longitudinal axis. In a holding state, the surface of the release element at the transition to the surface of a module housing may project by at most 2 mm, or by at most 1 mm, beyond the latter, or is recessed by at most by 2 mm, or at most by 1 mm, relative to the latter. In the mounted state, the surface of the release element may thus be an at least approximately seamless and flush continuation of a surface of the module housing of the add-on module, which surface may surround the release element directly. A handle or operating surface of the release element may not need to be completely flat or flush, but may also include an edge at the transition to a side surface of the module housing, although levers or rotary buttons protruding from the module housing as release elements may be excluded. The handle may be arranged or configured such that, when the handle is gripped in a first or injection grip position, a user grasps and touches, with a first hand, portions of the module housing and of the surface of the release element of the add-on module. In this grip position, all relevant manipulations may be carried out with the injection system—for example, placement on the injection site, injecting, and discharging by touching the discharge button with the thumb of the first hand. With the aid of the second hand, further manipulations such as removing the device cap, mounting an injection needle, and setting a dose may be carried out in the first grip position. Because the user may at least partially cover the release element of the add-on module with their hand in the first grip position, simultaneous actuation of the release element may be ruled out. To remove the add-on module, a second or release grip position may be required to be selected accordingly.

In addition or alternatively, the receiving region of the add-on module for receiving the injection device may be configured for inserting the injection device in the distal direction or by pushing the add-on module onto the injection device in the proximal direction. For this purpose, the injection device may include a distally directed stop face for limiting the mounting movement of the add-on module on the injection device in the proximal direction. This stop face may be formed at the transition between a distal carpule holder and a proximal device housing of the injection device. A dose selection button, and optionally also a dose display, at the proximal end of the injection device may not be touched or compressed by the add-on module, so that the receiving region may not have to be matched to the dimensions of the dose selection button, and it may thus have a greater length perpendicular to the longitudinal axis than the housing of the injection device.

In such examples, the injection device may include a proximally-directed holding surface, which may be formed on a recess or protrusion of the injection device and the surface may differ radially, or perpendicularly to the longitudinal axis, from a surface of the device housing of the injection device. A holding element of the holding mechanism of the add-on module may engage behind the holding surface in the mounted state and may establish a form-fit engagement with the holding surface, so that the add-on module may be held or secured against a separating movement in the distal direction and may transfer forces of the user to the injection device in the distal direction. The holding surface may be part of a recess, e.g., a notch or indentation, or part of a protrusion, e.g., a flange or a rib, in the surface of the device housing of the injection device. In some cases, the recess may not be part of a dose display window of the injection device for displaying a set dose, so that the dose display window is not covered by the add-on module, and the dose display may remain directly visible for the user. Due to the provided form-fitting engagement of the holding element, the add-on module may be locked at the injection device, and a force-fit with the surface of the injection device or even a plastic deformation of the surface of the injection device caused by claws on the add-on module may be prevented.

In implementations, the injection device may include a distal carpule holder for receiving a container and a proximal device housing for receiving a delivery mechanism, where the carpule holder may be reversibly and releasably connected or, after initial, one-time accommodation of the container, permanently and non-releasably connected to the device housing. The holding surfaces may be formed by protrusions and/or recesses on the carpule holder, so that only the carpule holder, but not the device housing with the delivery mechanism, requires replacement or be adapted for modified holding surfaces. In this case, protrusions on the carpule holder may be formed as bulges on a flange or flange section, which may project beyond an adjacent surface of the device housing, where the flange at the same time also may form the stop face for limiting the mounting movement of the add-on module on the injection device. The holding surfaces on the injection device may be formed by protrusions or recesses which may structurally differ from knobs of the injection device for fixing a device cap. The protrusions or recesses may be positioned axially such that the same device cap may be mounted with or without an add-on module; the knobs for fixing the device cap may not be used by the holding mechanism of the add-on module.

In addition or alternatively, the release element may snap or click into a stable release position at the end of the release movement. The release element may thus be let go of at the end of the release movement without thereby making the separating movement of the injection device and the add-on module impossible, or so that other grip positions may be selected for the separating movement. The release element may include two end positions; the release position of the release element may clearly differ visually from the holding position, which may result in a first and permanent holding state feedback for the user. Alternatively, the release element may be pretensioned by a spring mechanism into a holding position and may be required to be actively held in the release position by the user at least at the beginning of the separating movement.

In addition or alternatively, when the injection device is inserted, the release element may execute a holding or locking movement directed counter to the release movement, in which the holding element of the holding mechanism is moved in engagement with the holding surface, and the add-on module may be locked to the injection device. The holding element may be integrally formed with the release element or may be rigidly coupled at least in the direction of the holding/release movement. The release element may be indirectly moved for instance by a deflection mechanism or by unlocking a drive element from a released to a holding position via the axial insertion movement of the injection device, and no further locking element may need to be actuated by the user. The release element may therefore serve as a control (e.g., a release control device) and at the same time as a locking part.

In addition or alternatively, the holding mechanism may include a control element, which may be moved under tension of a control spring when the injection device is inserted, thereby releasing the release element from a release position to a holding position for the holding movement. The holding or locking movement may take place automatically, e.g., by means of an unlocked drive element such as a return spring, as a result of which a clearly audible locking click may be generated in a reproducible manner as an additional feedback of the holding state for the user.

In addition or alternatively, the injection device or a first injection device may include protrusions or recesses which may differ significantly from a surrounding surface of the injection device. The add-on module may include grooves corresponding to the protrusions in a lateral face of the receiving region, which may enable axial guidance of the protrusions or of the injection device during insertion into the add-on module, and/or holding elements, corresponding to the recesses, for engagement into the recesses. A further or a second injection device may differ from the aforementioned injection device solely by the protrusions or recesses and for instance may correspond to a medical indication such as a medical label or indication of use for the injection device. Differences in the protrusions or recesses result, for example, from the number, arrangement, and/or shape of discrete formations or indentations; differences in the medical indication may be based upon active ingredient, formulation, shelf life, use, therapy and/or patient, and the corresponding information on the label of the injection device. Individualized protrusions or recesses may prevent the add-on module from being mounted, intentionally or unintentionally, on the additional injection device that is identical except for the aforementioned differences.

In addition or alternatively, the add-on module may include a charging socket for connecting a charging cable for charging an electrical energy store of the add-on module. The charging socket may be arranged such that it is made inaccessible by the injection device when inserted; for instance the charging socket may open into the receiving region and may thus be directly covered by the injection device. The charging cable inserted into the charging socket may be guided out of the receiving region through a lateral opening or a window in the module housing, where the opening may be configured such that at least a portion of a label of an inserted injection device can be read through the opening.

In addition or alternatively, the carpule holder of the injection device and, optionally, an injection needle may be covered or protected between two or more injection processes by a device cap, which may be repeatedly removed and replaced. The add-on module may include a device cap detector for detecting the mounted device cap, with a tilting element comprising two legs originating from a pivot point, where a movement of a first leg end perpendicular to the longitudinal axis of the injection device may be converted or translated into an axial movement of a second leg end by a stroke corresponding to a wall thickness of the device cap. The movement of the second leg end and/or its end position may be detected by a switching element, which may respond to an axial movement, and which may indicate that the device cap has been properly mounted, e.g., completely and correctly.

In implementations, the add-on module may include an injection device detector for detecting the inserted injection device, with a switching element, which may be actuated by the release element in the locking movement and/or in the subsequent holding position. The release element may be configured as a release button with an axially-aligned lever and be pretensioned in a radial direction perpendicular to the longitudinal axis by a return spring for a holding or locking movement. The inserting injection device—optionally, via a linear or tilting movement of a control element—may release this locking movement, as a result of which the injection device is held in the add-on module, and the switching element may detect the movement of the release element and conclude that the injection device has been inserted completely and correctly.

Providing safe injection systems may also be achieved by providing an add-on module for an injection device having a longitudinal axis and a discharge button arranged at a proximal end of the injection device and movable in the distal direction along the longitudinal axis for discharging a liquid medication from a container of the injection device, where the add-on module includes a sensor unit, a receiving region adapted to a shape of a device housing of the injection device for being mounted on the injection device along the longitudinal axis, and a holding mechanism, which may releasably secure the mounted add-on module against moving axially relative to the injection device, where the receiving region may be configured such that the discharge button can be touched even when the add-on module is mounted, and where the add-on module comprises a release element, which in a release movement for releasing the holding mechanism, may be moved solely in a plane perpendicular to the longitudinal axis. The receiving region may be configured to receive the injection device by inserting the injection device in the distal direction, where the injection device has a holding surface directed proximally, which engages behind a holding element of the holding mechanism and holds the injection device proximally against a separating movement.

In implementations, providing safe injection systems including the injection device and add-on module releasably mounted thereon may be configured such that a user cannot recharge an electrical energy store of the add-on module during an injection process. This may be achieved by providing the add-on module with a sensor unit, a receiving region adapted to a shape of a device housing of the injection device for being mounted on the injection device, and a charging socket for connecting a charging cable to charge an energy store of the add-on module, where the charging socket is arranged so as to be made inaccessible as a result of an inserted injection device. As a result, proof of the protection of a user of the injection device from electrical shock such when the charging cable is inserted, which proof may otherwise be required by regulations, can be avoided. In particular, the charging socket may open into the receiving region of the add-on module and may thus be directly covered by the received injection device. The add-on module may include a lateral opening or a window in the module housing, which may not be used for inserting the injection device and through which a charging cable inserted into the charging socket can be guided out of the receiving region.

In implementations, providing safe injection systems including the injection device and add-on module releasably mounted thereon may be configured such that a correct execution of a mandatory action on the injection system may be communicated to a user. This may be achieved by providing the an add-on module with a sensor unit, a receiving region adapted to a shape of a device housing of the injection device for being mounted on the injection device, with a longitudinal axis and a removable device cap for covering a carpule holder, and with a device cap detector for detecting the mounted device cap, including a tilting element with two legs originating from a pivot point, where a movement of a first leg end perpendicular to the longitudinal axis of the injection device is converted or translated into an axial movement of a second leg end by a stroke corresponding to a wall thickness of the device cap, and including a switching element for detecting the axial movement of the second leg end and/or its end position. Complete and correct mounting of the device cap may be inferred from a corresponding signal of the actuated switching element, which may be suitably displayed to the user for confirmation and used for deactivating, for example, a communications unit.

In implementations, providing safe injection systems including the injection device and add-on module releasably mounted thereon may be configured such that a correct execution of a mandatory action on the injection system may be communicated to a user. This may be achieved by providing an add-on module with a sensor unit, a receiving region adapted to a shape of a device housing of the injection device for being mounted on the injection device, and a release element, which may be moved by a locking movement to a holding position in which the release element releasably secures the mounted add-on module against moving axially relative to the injection device, where the add-on module may include an injection device detector for detecting the inserted injection device, with a switching element, which may be actuated by the release element in the locking movement and/or in the holding position. Complete and correct insertion of the injection device may be inferred from a corresponding signal of the actuated switching element, which may be suitably displayed to the user for confirmation and for activating, for example, a communications unit.

In the aforementioned solutions, the electronic add-on module may be releasably mounted or pushed onto an injection device along the longitudinal axis, which may connect a distal end on the injection side to an opposite proximal end of the injection device. The injection device may be configured with a device housing, which may surround or enclose a delivery mechanism for parenteral subcutaneous or intramuscular delivery of medications from a container of the injection device. For this purpose, the injection device may include a discharge button at its proximal end, which may be moved or triggered by a user in the distal direction along a longitudinal axis of the injection device and may thereby cause or trigger a discharge. This discharge button may be directly touched and operated by the user even when the add-on module is mounted, and may not be surrounded or covered by the add-on module. The add-on module may include the sensor unit for detecting processes or states in the injection device, and a processor unit for evaluating and/or processing a signal of the sensor unit. The add-on module may further include a rechargeable energy store for supplying energy to the sensor unit and/or processor unit, e.g., a pouch cell, and a communications unit for wireless communication of signals from the sensor unit and/or of data of the processor unit. The add-on module may include a holding mechanism, which may releasably secure the mounted add-on module against at least axial movement relative to the injection device. For this purpose, the add-on module may include a release element or release control device, which may be operated by a user in a release movement for releasing the holding mechanism and for releasing the add-on module from the injection device.

Further embodiments and configurations are readily and clearly apparent to a person skilled in the art; they result from combinations of the described examples or from combining the described examples and the general technical knowledge of the person skilled in the art.

### BRIEF DESCRIPTION OF THE DRAWINGS

Implementations are described in connection with the appended figures, which are exemplary and are in no way to be interpreted as limiting. In the drawings:

FIG. 1 shows an injection device configured for variable dose delivery, according to the present disclosure;

FIGS. 2A and 2B show side and end views of a first variant of an add-on module with a sliding release element, according to the present disclosure;

FIGS. 3A and 3B show cross-sections of a first embodiment of the first variant of the add-on module configured for a force-fit engagement taken along line A-A of FIG. 2A, according to the present disclosure;

FIGS. 4A, 4B and 4C show three injection devices each with different protrusions or recesses;

FIGS. 5A and 5B show cross-sections of a second embodiment of the first variant of the add-on module configured with a form-fit engagement, according to the present disclosure;

FIGS. 6A, 6B, 6C and 6D show views of a third embodiment of the first variant of the add-on module configured with a form-fit engagement, according to the present disclosure;

FIGS. 7A and 7B show side and end views of a second variant of an add-on module configured with a rotating release element, according to the present disclosure;

FIGS. 8A and 8B show cross-sections of a first embodiment of the second variant of the add-on module configured with a form-fit engagement, according to the present disclosure;

FIGS. 9A and 9B show cross-sections of a second embodiment of the second variant of the add-on module configured with a form-fit engagement, according to the present disclosure;

FIGS. 10A and 10B show side and end views of a third variant of an add-on module with a push-button;

FIG. 11 shows a cross-section of a first embodiment of the third variant of the add-on module configured with a force-fit engagement, according to the present disclosure;

FIGS. 12A and 12B show cross-sections of a second embodiment of the third variant of the add-on module configured with a form-fit engagement, according to the present disclosure;

FIGS. 13A and 13B show end views of a third embodiment of the third variant of the add-on module configured with a form-fit engagement, according to the present disclosure;

FIGS. 14A and 14B show isometric and cross-section views of a fourth embodiment of the third variant of the add-on module configured with a form-fit engagement, according to the present disclosure;

FIGS. 15A and 15B show cross-sections of a first implementation with a form-fit engagement against protrusions;

FIGS. 16A and 16B show cross-sections of a second implementation with a form-fit engagement against recesses;

FIGS. 17A and 17B show partial cross-sections of a fifth embodiment of the third variant of the add-on module configured with a form-fit engagement, according to the present disclosure;

FIGS. 18A and 18B show two, partial longitudinal sections of a sixth embodiment of the third variant of the add-on module configured with a form-fit engagement, according to the present disclosure;

FIGS. 19A, 19B, 19C, 19D, 19E, 19F, 19G, and 19H show several different versions of protrusions and recesses; and

FIG. 20 shows an add-on module with an inserted charging cable.

### DETAILED DESCRIPTION

The term, “medication” or “medical substance,” includes any flowable medical formulation which is suitable for controlled administration by means of a cannula or hollow needle—for example, a liquid, a solution, a gel, or a fine suspension containing one or more medical active ingredients. A medication can thus be a single active ingredient composition or a pre-mixed or co-formulated composition having several active ingredients from a single container. The term includes in particular drugs such as peptides (e.g., insulins, insulin-containing medications, GLP-1-containing and derived or analogous preparations), proteins and hormones, biologically-obtained or active ingredients, active ingredients based upon hormones or genes, nutrient formulations, enzymes, and further substances, both in solid (suspended) or liquid form. The term furthermore also includes polysaccharides, vaccines, DNA or RNA or oligonucleotides, antibodies or parts of antibodies, and suitable base, auxiliary, and excipient substances.

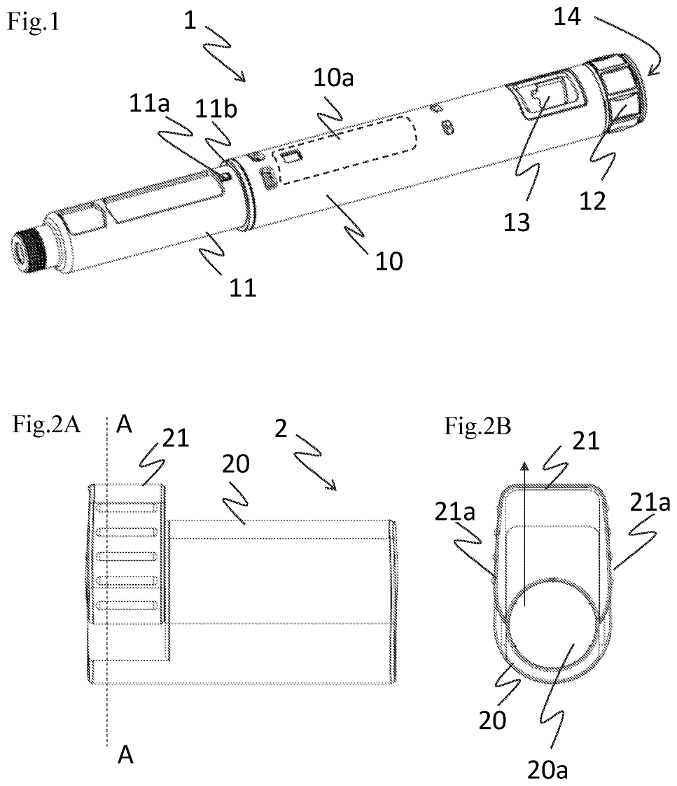

FIG. 1 shows an isometric view of an injection device 1 with variable dose delivery, including an elongate device housing 10 with a longitudinal axis, a carpule holder 11, a dose selection button 12, a dose display 13, and a discharge button 14. The carpule holder 11 may include a cam 11a and an annular stop face 11b at the transition to the device housing 10. A distal end of the carpule holder 11 (on the left in FIG. 1) may include a thread for receiving a needle unit with an injection needle. A device or needle shield cap of the injection device 1 may engage in a snap connection with the cam 11a for covering the carpule holder 11. A possible area of a bearing face 10a on the upper side of the device housing 10 is shown by a dashed line. A label of the injection device may be applied to a lower side of the device housing 10 opposite the bearing face 10a.

FIGS. 2A and 2B show a first variant of an add-on module 2, according to the present disclosure, in a side view perpendicular to the longitudinal axis (FIG. 2A) and in an end view in the direction of the longitudinal axis (FIG. 2B) in a released state. The add-on module 2 may include a sleeve-shaped module housing 20 with a receiving region 20a for receiving the injection device from FIG. 1 by inserting the injection device 1 in the distal direction, or by pushing the add-on module 2 onto the injection device 1 in the proximal direction up to the stop with the stop face 11b. In the mounted state, a lateral face of the receiving region 20a of the module housing 20 may rest tightly at least against the bearing face 10a of the device housing 10. The add-on module 2 may also include a release element 21 configured as a slide, which may be moved in the direction of the upwards arrow (FIG. 2B), perpendicular to the longitudinal axis, from a holding state to the released state shown. The release element 21 may include two lateral gripping surfaces 21a, each having five ribs, which in the direction of the longitudinal axis, may include a length corresponding to the width of a finger. As a result, the release element 21 may be gripped by the thumb and forefinger of one hand and moved, e.g., pushed, perpendicular to the longitudinal axis. In the released state shown, the release element 21 may project beyond the module housing 20, whereas, in a holding state, the release element 21 and the module housing 20 may form a continuous surface, for instance without noticeable shoulders or transitions, which may be suitable for use as a handle for a user to grip the add-on module and injection device.

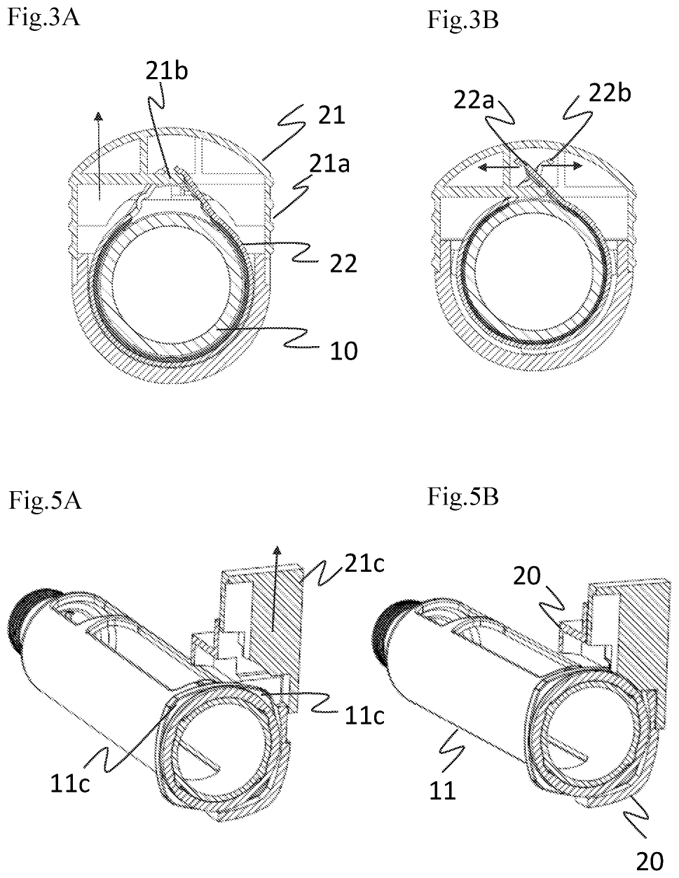

FIGS. 3A and 3B show cross-sections of the device housing 10 and module housing 20 perpendicular to the direction of the longitudinal axis and in a plane A-A of FIG. 2A, according to a first embodiment of the first variant of the present disclosure, in which the add-on module 2 is held on or fastened to the device housing 10 of the injection device 1 by means of a force-fit engagement. A metal clamp 22 of the add-on module 2 may be constructed of a metal strip with a width that does not exceed the axial length of the release element 21, and a length that does not fall below the circumference of the device housing 10. The metal strip may form a loop with a 270° curved section and two, partially intersecting ends 22a, 22b, each with a width of less than half the width of the strip, which may be offset in the axial direction. In the holding state (FIG. 3B), the curved section may rest tightly against the device housing 10, and the two ends 22a, 22b may be pretensioned crosswise in the opposite pretensioning directions, indicated by the two arrows. A force-fit engagement with a minimum static friction may be established between the device housing 10 and the curved section, and optionally via a radially-compressible section, of the module housing 20. This may prevent unintentional release of the add-on module 2 counter to the mounting direction, as may possibly be caused by axial forces exerted on the injection device 1 when a needle shield cap is mounted or a needle unit applied.

In a released state (FIG. 3A), the two ends 22a, 22b of the metal strip may be moved away from each other or spread counter to their respective pretensioning directions, and the curved section of the metal strip may rest less tightly against the device housing 10 when the force-fit engagement is released, so that the device housing 10 is movable relative to the add-on module 2. The movement of the two ends 22a, 22b may be controlled by a release wedge 21b of the release element 21—in the present case, by a web extending in the longitudinal direction, which may separate the two ends upwardly in the direction of the arrow during a release movement. If the metal clamp 22 is not pretensioned or pretensioned in a different direction, the release element 21 may compress the two ends 22a, 22b of the metal strip, even in the holding position. The clamp 22 may also include several windings from a correspondingly less wide strip, or may be constructed of wire.

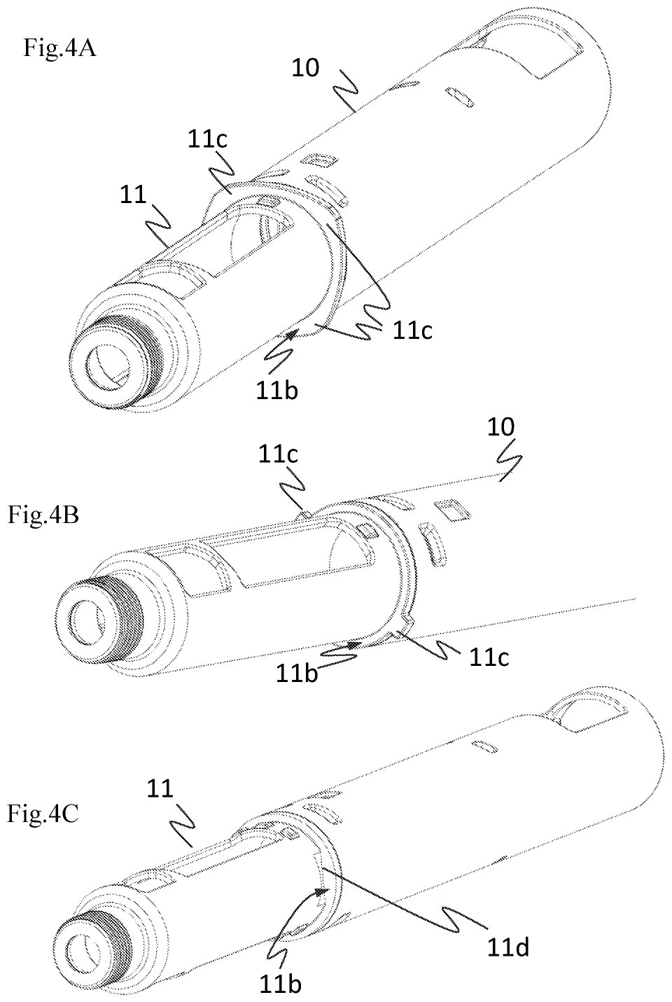

FIGS. 4A, 4B and 4C show three injection devices 1 with different protrusions or recesses on the carpule holder 11 for use in establishing the form-fit engagement of an add-on module 2. In FIG. 4A, the protrusions 11c may have the shape of four radial extensions, widenings, or prominences of a flange which are distributed evenly over the circumference, which flange may also form the annular stop face 11b. In FIG. 4B, the protrusions 11c may have the shape of two lateral cams on opposite sides of the flange with respect to the longitudinal axis. The protrusions 11c may extend beyond the surface of the device housing 10 and may thus form proximally-directed holding surfaces for establishing a form-fit engagement with a holding element of the add-on module 2 and securing against unintentional release of the add-on module 2 counter to the mounting direction. The protrusions at FIG. 4A may include less than four and/or non-rotationally-symmetrically-arranged extensions, or may also be configured as a circumferential collar. FIG. 4C shows recesses 11d on the carpule holder 11 in the form of two parallel, lateral notches or slots. The two notches may be arranged distal to the flange, and may include holding surfaces extending inwardly relative to the surface of the carpule holder 11 and may be directed proximally for a form-fit engagement with a holding element of the add-on module 2. The protrusions and recesses may also be formed on the device housing 10 or further distally of the stop face 11b on the carpule holder 11. A suitably configured add-on module 2 may include grooves in the receiving region 20a that correspond to the protrusions 11c and extend parallel to the longitudinal axis, which grooves, in the case of protrusions on the flange, may extend almost over the entire length of the receiving region 20a. As a result, the injection device 1 and the add-on module 2 may be secured in a torque-proof manner against each other during mounting.

FIGS. 5A and 5B show a second embodiment of the first variant with a form-fit engagement with two of four protrusions 11c of the carpule holder 11, for instance as shown at FIG. 4A. FIGS. 5A and 5B show a section perpendicular to the longitudinal axis directly proximal of the flange, where the module housing 20 is, additionally, halved in the axial direction. The release element 21 may include a radially-displaceable holding element 21c, the innermost edge of which in the holding state (FIG. 5B) may engage behind the holding surfaces of two of the four protrusions 11c and, after the release movement has been completed, releases them in the direction of the arrow in the released state (FIG. 5A).

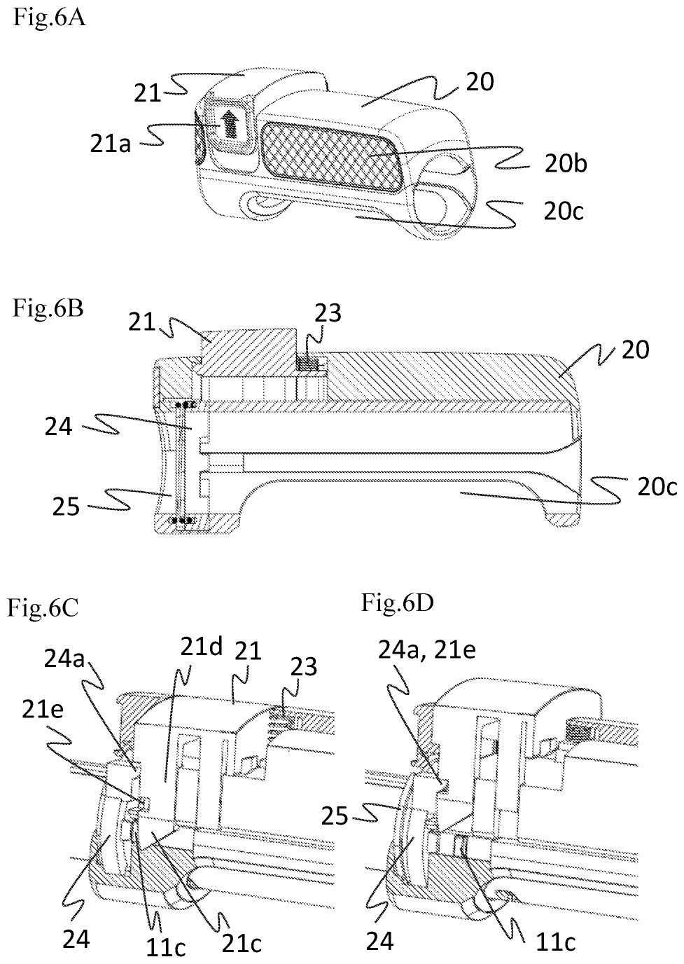

FIG. 6A shows, in an isometric view, a third embodiment of the first variant of the present disclosure configured for a form-fit engagement with the two lateral protrusions 11c on the flange of the carpule holder 11 according to FIG. 4B. The add-on module 2, in turn, may include a release element 21 in the form of a slider. In the area of a label of the injection device 1, e.g., opposite the bearing face 10a of the device housing 10, the module housing 20 may include a window 20c or an opening to the injection device. The module housing 20 may also include touch sensors 20b as provided herein. FIG. 6B shows a longitudinal section through the third embodiment in a released state. The radially-movable release element 21 may be pretensioned downwards or inwards by a return spring 23, and an axially-movable control element 24 may be pretensioned proximally by a control or ejection spring 25. The return spring 23 and the control spring 25 are shown as pressure-loaded compression springs; however, pretensioning of the release element 21 and the control element 24 may also be established by a tension spring or other elastic element.

FIGS. 6C and 6D shows a release element 21 with two parallel arms and a holding element on each arm, where only the arm 21d facing the observer is visible in the selected view. In the released state (FIG. 6D), a locking cam 24a of the control element 24 is engaged in a recess 21e of the release element 21, so that the release element 21 is blocked against movement downwards despite being pretensioned by the return spring 23. As soon as the injection device is inserted from the proximal direction and pushes the control element 24 distally, with the stop face 11b or the protrusions 11c under compression of the control spring 25, the locking cam 24a is pushed axially out of the recess 21e, and the release element 21 is released. The release element 21 is pushed downwards by the return spring 23 and, in the holding state, engages with its holding elements 21c behind the lateral protrusions 11c on the injection device (FIG. 6C). The engagement of a distally-directed holding surface of the holding element 21c behind the proximally-directed holding surfaces of the protrusions 11c on the injection device locks the add-on module 2, relative to the injection device 1, in the holding state. To release the add-on module 2, the release element 21 may be pulled upwards, away from the longitudinal axis at its lateral gripping surfaces 21a until locking by the holding elements 21c is released, and the locking cam 24a snaps into place again. The control spring 25 may relax and push the injection device out of the add-on module.

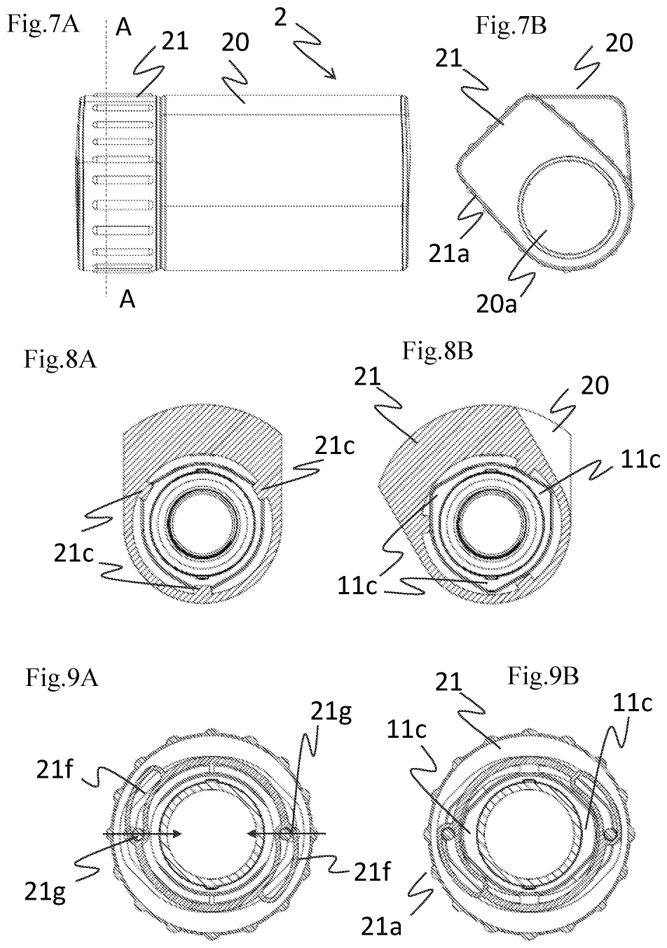

FIGS. 7A and 7B show a second variant of an add-on module 2 according to the present disclosure in a side view perpendicular to the longitudinal axis (FIG. 7A) and in an end view in the direction of the longitudinal axis (FIG. 7B) in a released state. The add-on module 2 may include a sleeve-shaped module housing 20 with a receiving region 20a for receiving the injection device of FIG. 1 by inserting the injection device 1 in the distal direction or by pushing the add-on module 2 onto the injection device 1 in the proximal direction up to the stop with the stop face 11b. In the mounted state, a lateral face of the receiving region 20a rests tightly at least against the bearing face 10a of the device housing 10. The add-on module 2 may include a release element 21 configured as a control lever, which may be rotated or turned in a plane perpendicular to the longitudinal axis from a holding state to the released state. The release element 21 may include a gripping surface 21a on all sides with parallel ribs which, in the direction of the longitudinal axis, and may have a length corresponding to the width of a finger. As a result, the release element 21 may be gripped by the thumb and forefinger of one hand and be rotated, turned, tilted, or pivoted perpendicularly to the longitudinal axis. In the holding state, the release element 21 and the module housing 20 may form a continuous surface without noticeable shoulders or transitions, which may be suitable as a handle for a user to grip the add-on module and injection device.

FIGS. 8A and 8B show cross-sections perpendicular to the direction of the longitudinal axis taken along plane A-A of FIG. 7A, and, distally, in the direction of view, a first embodiment of the second variant with a form-fit engagement against three protrusions 11c on the carpule holder 11. At least the tips of the protrusions 11c may project radially beyond a device housing 10 or carpule holder 11 surface surrounding them. The release element 21 may include three, inwardly-pointing holding elements 21c in the form of projections, which may be distributed over an inner circumference in accordance with the arrangement of the protrusions 11c and may be connected to the release element 21 in a rotationally-fixed manner and for instance the release element 21 may be configured as an adjusting ring. In the holding state (FIG. 8A), the holding elements 21c may be in a form-fitting relationship to the protrusions 11c, and may thus prevent a movement of the add-on module 2 in the distal direction. Through a rotation by at most 60°, the protrusions 11c may be released by the release element 21 for a movement in the proximal direction (FIG. 8B).

FIGS. 9A and 9B show cross-sections perpendicular to the direction of the longitudinal axis in the direction of view distal of a first embodiment of the second variant with a form-fit engagement against two protrusions 11c on the carpule holder 11. In this embodiment, the release element 21 may be configured as a circular adjusting ring with a circumferential gripping surface 21a. FIG. 9B shows a released state, while FIG. 9A shows a holding state. During the transition from the released to the holding state, two radially-movable-mounted guide cams 21g may be pushed inwards in the direction of the arrow by a 45° clockwise rotation of the adjusting ring and by means of two eccentric guide notches 21f coupled to the adjusting ring in a rotationally-fixed manner. The guide cams 21g may be coupled to radially-movable holding elements at least in the radial direction or may be formed integrally therewith, so that the holding elements also move inwards and engage behind the protrusions 11c. A rotational movement of the release element 21 may also be implemented or deflected by another slide control system or a wedge into the radial movement of a holding element instead of by the guide notches, where the guide cams may also be attached in a radially-fixed manner to the adjusting ring and may be provided on the holding element for interacting with an eccentric guide notch.

Further embodiments of the second variant relate to a combination of a tilting or rotating release element, as shown herein, with a force-fit engagement on the device housing 10. A clamping force necessary for this engagement may be produced by a pretensioned metal strip, as described in connection with FIGS. 3A and 3B, where an end of the metal strip with the release element may be rotated against the other end of the metal strip that is connected to the module housing, in order to release the force-fit engagement. Alternatively, brake shoes, e.g., in place of the holding elements from FIGS. 9A and 9B, may be pressed radially onto the surface of the housing of the injection device 1 via guide cams and eccentric guide notches.

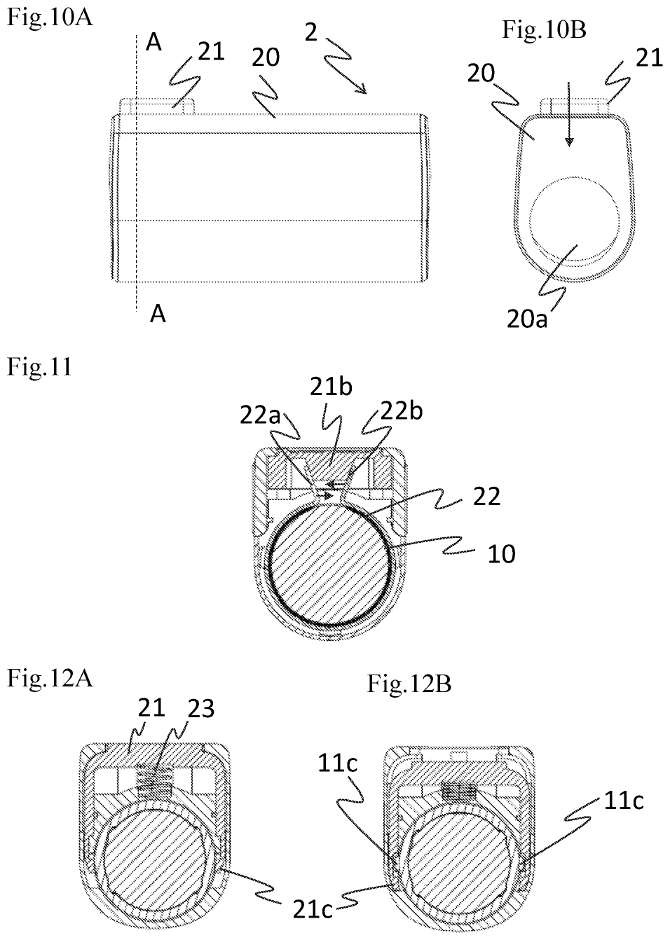

FIGS. 10A and 10B show a third variant of an add-on module 2 according to the present disclosure in a side view perpendicular to the longitudinal axis (FIG. 10A) and in an end view in the direction of the longitudinal axis (FIG. 10B) in a released state. The add-on module 2 may include a sleeve-shaped module housing 20 with a receiving region 20a for receiving the injection device from FIG. 1 by inserting the injection device 1 in the distal direction or by pushing the add-on module 2 onto the injection device 1 in the proximal direction up to the stop at the stop face 11b. In the mounted state, a lateral face of the receiving region may rest tightly at least against the bearing face 10a. The add-on module 2 may also include a release element 21 configured as a push button, which may be moved perpendicularly to the longitudinal axis from a holding state to the released state shown. The release element 21 may include an upwardly-directed square or round pressure receiving or operating surface with a length corresponding to a fingertip of a user—such as at least 0.5 cm². As a result, the release element 21 may be pressed downwards in the direction of the arrow perpendicular to the longitudinal axis in a release movement. In the holding state, the release element and the module housing 20 may form a continuous surface without noticeable shoulders or transitions, which may provide a handle or grip for a user to grip the add-on module and injection device. In this case, the pressure receiving or operating surface of the release element 21 may differ from the planar shape shown and may also include an edge at the transition to an inclined, side surface section of the module housing 20. In a released state, the release element 21 for instance configured as the push button may project beyond the module housing 20 as shown, may be recessed in relation to the surface of the module housing, or may, as a push button, also assume the same position as in the holding state.

FIG. 11 shows a cross-section perpendicular to the direction of the longitudinal axis in a plane A-A according to FIG. 10A of a first embodiment of the third variant with a force-fit engagement, according to the present disclosure. A metal clamp 22 may be formed from a metal strip with a width, which may not exceed the axial length of the release element 21, and with a length which may not fall below the circumference of the device housing. The metal strip may form a loop with a 320° curved section and two ends 22a, 22b angled therefrom. In the holding state shown, the curved section rests tightly against the device housing 10, and the two ends 22a, 22b are pretensioned or pushed in the opposite pretensioning directions that are indicated by the two arrows. A force-fit engagement with minimum static friction may be produced between the device housing 10 and the metal strip—optionally, via a radially-compressible section of the module housing 20.

In a released state, the two ends 22a, 22b of the metal strip may be moved away from each other or spread apart counter to their respective pretensioning direction, and the curved section of the metal strip may rest less tightly against the device housing 10 when the force-fit engagement is released, so that the latter is movable relative to the add-on module 2. Movement of the two ends 22a, 22b may be controlled by a release wedge 21b of the release element 21, and in the present case by a wedge-shaped web extending along the longitudinal direction, which may separate the two ends 22a, 22b counter to their pretensioning when a user presses the release element 21, and the latter executes a release movement in the radial direction.

FIGS. 12A and 12B show cross-sections perpendicular to the direction of the longitudinal axis in a plane A-A according to FIG. 10A in the distal direction of view of a second embodiment of the third variant with a form-fit engagement against two lateral protrusions on the carpule holder 11 according to FIG. 4B. The release element 21 may include two parallel arms at a distance corresponding to the diameter of the injection device. Included at the end of each arm may be a holding element 21c, which may engage behind one of the protrusions 11c on the carpule holder 11 in the holding position (FIG. 12A). Upstream of the holding elements 21c, release elements or recesses in the arms may be provided, which, in the released state (FIG. 12B), may be aligned with the protrusions 11c after a pressing movement to release the protrusions 11c. The release element 21 may be pretensioned by a return spring 23 into the holding position, which may be compressed during a release movement to the released state.

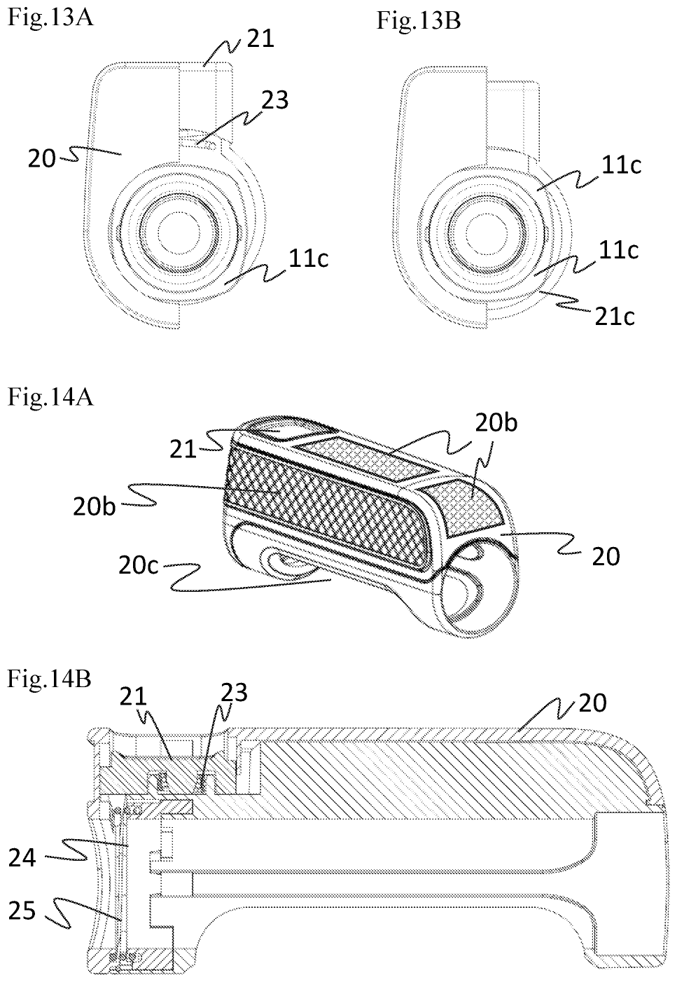

In the proximal direction of view, FIGS. 13A and 13B show cross-sections perpendicular to the direction of the longitudinal axis in a plane A-A according to FIG. 10A according to a third embodiment of the third variant with a form-fit engagement against four protrusions on the carpule holder according to FIG. 4A. One half of the module housing 20 and the push button of the release element 21 are shown in an axial view. The flange on the carpule holder 11, in turn, includes four protrusions 11c that are evenly distributed. The release element 21 may include a holding element 21c, which may engage behind two protrusions 11c on a side of the injection device opposite the push button when the release element 21 is pushed into the holding position (FIG. 13A) by the return spring 23.

FIGS. 14A and 14B show a fourth embodiment of the third variant with a form-fit engagement to the injection device, according to the present disclosure. The add-on module 2 shown at FIG. 14A in an isometric view, includes a release element 21 in the form of a push button as well as touch sensors 20b described further herein. In the area of a label of the injection device, the module housing 20 has a window 20c or an opening, e.g., opposite the bearing face. FIG. 14B, shows a longitudinal section of FIG. 14A in a released state. The upwardly-directed pressure receiving or operating surface of the release element 21 is shown as lowered relative to a surface of the surrounding module housing 20, e.g., by at least 1 mm. The release element 21 may be pretensioned upwards by a return spring 23 counter to the direction of pressure exerted by the user, and an axially-movable control element 24 may be pretensioned proximally by an ejection or control spring 25. The control element 24 may be annular and the injection device may be inserted therethrough. The return spring 23 and the control spring 25 are shown as pressure-loaded compression springs; however, pretensioning of the release element 21 and the control element 24 may also be accomplished by a tension spring or other elastic element.

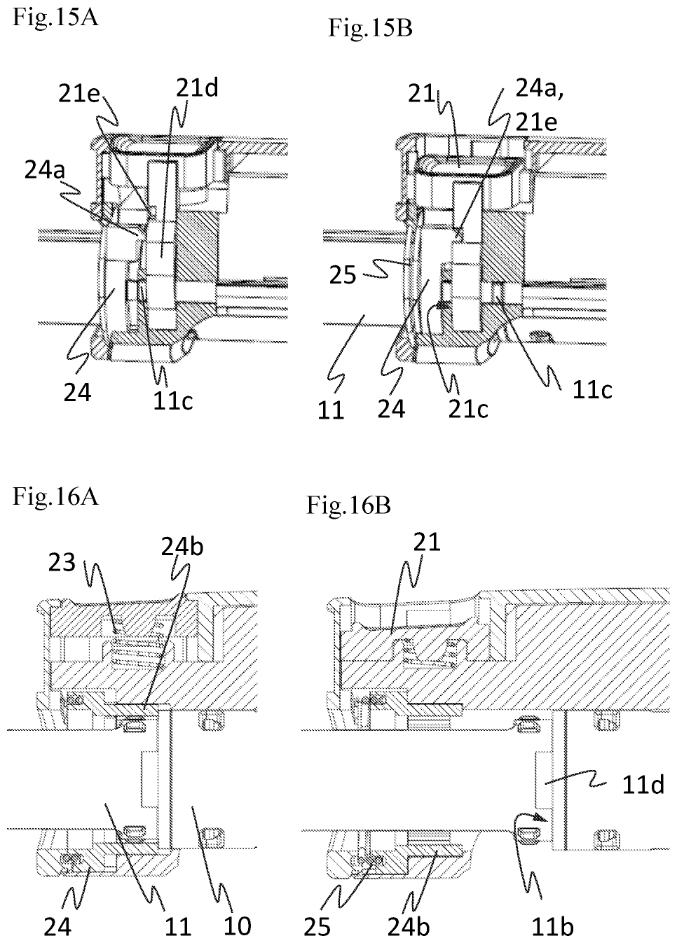

FIGS. 15A and 15B show a first implementation of the fourth embodiment of the third variant according to the present disclosure with a form-fit engagement against two lateral protrusions 11c of the carpule holder 11 according to FIG. 4B. The release element 21 may include two parallel arms and a holding element on each arm, where, as shown, only the arm 21d facing the observer is visible, and the associated holding element 21c is located on the inside of the arm 21d. In the released state (FIG. 15B), a protrusion in the form of a locking cam 24a of the control element 24 may be engaged in a recess 21e of the release element 21, so that the latter is blocked against movement upwards, despite being pretensioned by the compression spring 23. As soon as the injection device is inserted from the proximal direction and pushes the control element 24 distally with the stop face 11b or the protrusions 11c, under compression of the control spring 25, the locking cam 24a may be pushed axially out of the recess 21e, and the release element 21 may thus be released. The release element 21 may be pushed upwards by the return spring 23 and engage with its holding elements 21c behind the lateral protrusions 11c on the injection device (FIG. 15A). As a result, the add-on module 2 may be locked in the holding state relative to the injection device.

To release the add-on module 2, the release element 21 may require being pressed downwards by pressing the pressure surfaces towards the longitudinal axis until the locking is released by the holding elements 21c, or release elements or recesses in the arms are aligned with the protrusions 11c, analogously to the embodiment according to FIGS. 12A and 12B. The locking cams 24a may snap into place again, and the control spring 25 may relax and push the injection device out of the add-on module 2. Alternatively, the locking cam 24a may also be provided on the release element 21 and engage in a recess of the control element 24. For tolerance reasons, in the holding state, further compression of the control spring 25 or a small axial movement of the injection device, of its protrusions, and of the control element 24 in the distal direction relative to the holding elements 21c of the release element 21, and thus to the module housing 20, may not be ruled out. In accordance with the present disclosure, however, even when such movement is present, with a tolerance-related or intended path or movement between one tenth and one millimeter, the add-on module is considered to be axially held relative to the injection device.

FIGS. 16A and 16B show a second implementation of the fourth embodiment of the third variant of the present disclosure with a form-fit engagement against recesses on the carpule holder in the form of two, parallel, lateral notches according to FIG. 4C. The two notches 11d are arranged after the flange in the distal direction and replace the lateral protrusions of the first implementation. As shown in the longitudinal sections in FIGS. 16A and 16B in the released state (FIG. 16B), and as further differing from the first implementation, the control element 24 includes a proximally-directed extension 24b in the form of several plungers or pins. A proximal end of the extension 24b strikes the stop face 11b on the carpule holder 11 of the inserting injection device and pushes the control element 24 distally. In the holding state (FIG. 16A), locking cams of the control element 24 release the release element 21 for an upwards movement, as a result of which holding elements of the release element 21 are inserted into the notches. Due to the extension 24b, the movements of the control element and of the release element can be easily coordinated with one another; for example, it may be ensured that the holding element 21c is released for the locking movement only when the notch 11d has advanced sufficiently in the distal direction.

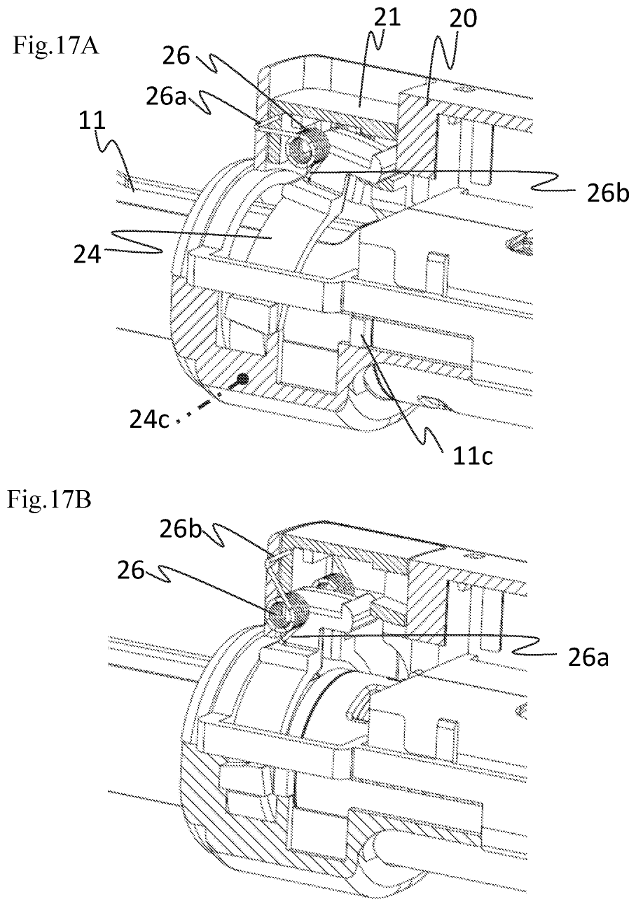

FIGS. 17A and 17B show longitudinal views of a fifth embodiment of the third variant with a form-fit engagement against two lateral protrusions of a carpule holder according to FIG. 4b in accordance with the present disclosure. In contrast to the fourth embodiment of FIGS. 15A and 15B, in this embodiment, the return spring and the control spring are combined in a single spring, and the control element is mounted in a rotatable instead of in an axially-displaceable manner. As shown in the released state at FIG. 17A, the control element 24 has an axis of rotation 24c, with which the control element 24 is rotatably anchored in the module housing 20, and about which it can be tilted from the release position to a holding position (FIG. 17B) when the injection device is pushed distally relative to the module housing 20. For this purpose, the opening for the injection device formed by the control element 24 may be configured to be slightly oval in a direction perpendicular to the axis of rotation 24c. The release button 21, in turn, may include two, lateral, parallel arms with recesses into which locking cams of the control element 24 may engage in the release position, and with holding elements which, in the holding state, may engage behind the protrusions 11c. A leg spring 26 may press the push button of the release element 21 upwards with a first leg 26a, and press the control element 24 in the proximal direction with a second leg 26b. In the release position, the pressure surface of the release element 21 may be recessed, and the leg spring 26 may be tensioned. In the holding position, the control element 24 may be oriented to be perpendicular to the longitudinal axis, the pressure surface of the release button 21 may be flush with the surface of the module housing 20, and the leg spring 26 may be slightly relaxed, since a tensioning movement of the first leg 26a may be exceeded by a relaxation of the second leg 26b. In this embodiment, too, by pressing the release button 21, the locking of the protrusions 11c may be released, the injection device may be pushed proximally, and the release button 21 may be locked in the pushed-in release position (FIG. 17A).

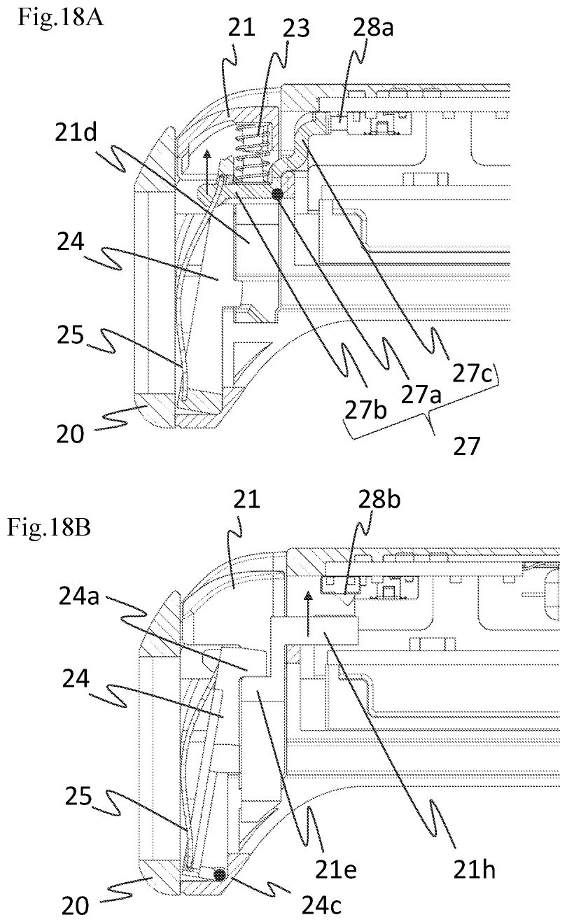

FIGS. 18A and 18B show partial longitudinal cross-sections of a sixth embodiment of the third variant with a form-fit engagement against two lateral protrusions of a carpule holder according to FIG. 4B, in a release position according to the present disclosure. As in the preceding fifth embodiment, the control element 24 may be rotatably mounted and, as in the fourth embodiment of FIGS. 15A and 15B, the return spring 23 and the control spring 25 may be separated and thus individually adjustable in this embodiment. As shown at FIG. 17A, the control element 24 in FIGS. 18A and 18B also has an axis of rotation 24c in a lower side, opposite the release button 21, with which it is rotatably anchored in the module housing 20, and about which the control element 24 may be tilted from the release position shown, to a holding position, when the injection device is pushed distally relative to the module housing 20. The control spring 25 may be a non-planar spring ring or a spring clip with an opening for the injection device, which may be supported, in each case on two points, opposite one another with respect to the longitudinal axis, on the module housing 20 or on the control element 24. The release button 21, in turn, may include two, lateral, parallel arms 21d with recesses 21e into which locking cams 24a of the control element 24 may engage in the release position, and with holding elements which, in the holding state, may engage behind the protrusions 11c of the injection device. The return spring 23 may be a pressure-loaded compression spring.

FIG. 18A shows a device cap detector with a tilting element 27, which is configured to tilt about a pivot point 27a as soon as the device cap, in the proximal direction (from the left in FIGS. 18A and 18B), is either mounted on the carpule holder 11 of the injection device inserted in the add-on module 2, or is removed therefrom. A first or distal leg 27b of the tilting element may be held in an approximately axial orientation by the return spring 23 and may be pushed upwards (in the direction of the arrow) from this position by a proximal edge of the device cap. As a result, one end of a second or radial leg 27c of the tilting element 27, which is arranged at an angle of approximately 90° relative to the first leg, may be moved substantially in the proximal direction, which may be detected by a switching element 28a actuated in the axial direction.

FIG. 18B, shows an injection device detector, which may include an axially-aligned lever 21h on the release button 21, and a switching element 28b. Due to an inserted injection device, the release button 21 may, as described, be released for a radial movement perpendicular to the longitudinal axis (e.g., upwards in FIG. 18B); the corresponding movement or end position of the lever 21h may be detected by the switching element 28b. The presence of the injection device may thus be detected only at the very end of the insertion movement, and not only after partial insertion. The injection device detector may be provided alternatively or additionally to the device cap detector. The switching elements 28a, 28b may include electronic micro switches, sliding contacts, or optical detectors; which may be arranged on a printed circuit board fixedly mounted in the module housing 20.

FIGS. 19A-19E show five different versions of the protrusions 11c on the flange of the carpule holder, starting with the version of FIG. 4B as FIG. 19A. The five versions of the protrusions 11c differ with respect to the number of protrusions, their arrangement or distribution over the circumference of the carpule holder, and their shape in the shown sectional plane perpendicular to the longitudinal axis. The shape includes both a width or length in the circumferential direction, a height or extension in the radial direction, as well as a contour of the individual protrusions, and, for instance, differ from a perfectly radial or circular contour section. In some implementations, however, no protrusion is provided in the angular range of the bearing face on the injection device, e.g., over a corresponding angular segment of at least 120°, or at least 90°. As a result, the lateral face of the receiving region, which rests tightly against the bearing face in the holding state is not impaired by a groove for axial guidance of a protrusion.

FIGS. 19F-19H show in cross-sections perpendicular to the longitudinal axis, three different versions of recesses 11d on the carpule holder 11, together with the release element 21 of an add-on module 2 suitable to receive the carpule holder 11 and its recesses 11d. The release element 21 may form an opening for the injection device and may function with a return spring analogous to the embodiment of FIGS. 12A and 12B. The release element 21 may include a section opposite the push button with inwardly-directed protrusions as holding elements for engaging the recesses 11d of the carpule holder 11.

An add-on module, which corresponds or is adapted to one of FIGS. 19B-19E, cannot be mounted on an injection device with one of the other versions. At best, an injection device, with the configuration of FIG. 19A, may be inserted into an add-on module 2 for the FIG. 19B. Thus, in the sense of a key-lock principle, unambiguous assignments of injection devices to add-on modules may be generated. Individualized protrusions or recesses may prevent an add-on module from being intentionally or, if, for example, several injection devices are being used in the surroundings of a patient, accidentally mounted on a wrong injection device. A wrong injection device in the present context is an injection device which, except for the mentioned differences in the protrusions or recesses, is identical, but is provided for another medical indication. Differences in the medical indication include different active substances, formulations, shelf lives, therapies, and/or patients, and the corresponding information on the label. The protrusions may, in turn, also be arranged on the housing of the autoinjector or at another location of the carpule holder, where at least the axial arrangement of those protrusions, which are not engaged from behind by a holding element of the release element, can, for purely axial insertion movements, be arbitrary. In general, the mounting of an unsuitable add-on module may already be prevented, depending, however, upon the axial arrangement of the protrusions or, in the case of recesses, even the movement of the holding elements during an attempted transition to the holding state.

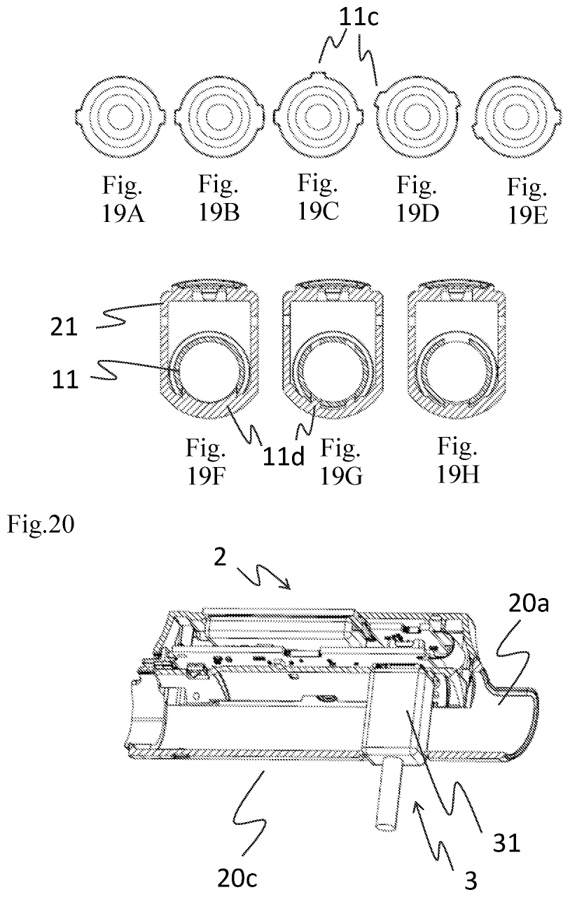

FIG. 20 shows a longitudinal cross-section through an add-on module 2 with an inserted charging cable 3 for charging or recharging an energy store of the add-on module 2 and/or for data exchange with a third-party device. The add-on module 2 may include a charging socket for receipt of a charging plug 31 of the charging cable 3, e.g., a USB-C port, which may be accessible solely from the receiving region 20a, or may be contacted solely by a user with a standard charging cable. Accordingly, the charging socket may be covered by an injection device inserted in the receiving region, and may thereby ensure that a charging cable must be removed again before the add-on module 2 is put into operation or before the injection device is inserted therein. The charging plug 31 may be inserted in a straight line through the opposite window 20c of the module housing 20 and transversely through the receiving region 20a. The size of the window 20c may correspond at least to a readable area of a label on the injection device and may thus provide sufficient space for manipulation of the charging cable—e.g., for gripping the charging plug 31 with two fingers. An alternative solution for preventing insertion of the charging plug 31 when the injection device is inserted may include movement of the charging socket at least partially behind an aperture in the surface of the module housing 20 when the injection device is inserted, or a bi-stable cover which, in order to release the charging socket, may be moved in front of an axial access to the receiving region solely and only when the injection device is missing from the add-on module 2.

As shown at least at FIG. 14A, the add-on module 2 may include touch sensors 20b. These touch sensors 20b may serve to detect when the add-on module is touched and for instance gripped. In the mounted state, an upcoming manipulation may inferred therefrom, and a sensor state, for example, may be activated. For this purpose, any conceivable possibility of gripping the add-on module may be reliably detected, e.g., not only a grip with the whole hand, but for instance also a grip in the manner of a pair of tweezers, with only two fingers on opposite sides of the add-on module. The add-on module may therefore include sensor regions on surfaces of both sides, on the top surface, and on the end faces. Exemplary capacitive touch sensors may be preferably affixed as a label to the module housing or encased as a film with the material of the module housing in an injection molding process. In addition to a touch sensor, the device cap detector from FIGS. 18A and 18B herein may also be used for detecting a mounted or removed device cap for an anticipated change of state of the add-on module.

LIST OF REFERENCE SIGNS

| | |
|---|---|
| 1 | Injection device |
| 10 | Device housing |
| 10a | Bearing face |
| 11 | Carpule holder |
| 11a | Cam |
| 11b | Stop face |
| 11c | Protrusion |
| 11d | Recess |
| 12 | Dose selection button |
| 13 | Dose display |
| 14 | Discharge button |
| 2 | Add-on module |
| 20 | Module housing |
| 20a | Receiving region |
| 20b | Touch sensor |
| 20c | Window or opening |
| 21 | Release element |
| 21a | Gripping surface |
| 21b | Release wedge |
| 21c | Holding element |
| 21d | Arm |
| 21e | Recess |
| 21f | Guide notch |
| 21g | Guide cam |
| 21h | Lever |
| 22 | Metal clamp |
| 22a, b | Ends |
| 23 | Return spring |
| 24 | Control element |
| 24a | Locking cam |
| 24b | Extension |
| 24c | Axis of rotation |
| 25 | Control spring |
| 26 | Leg spring |
| 26a, b | Spring leg |
| 27 | Tilting element |
| 27a | Pivot point |
| 27b, c | Lever leg |
| 28a, b | Switch |
| 3 | Charging cable |
| 31 | Plug |

What is claimed is:

1. An injection system, comprising:
an injection device having a longitudinal axis and comprising a discharge button arranged at a proximal end thereof, the discharge button movable in a distal direction along the longitudinal axis for discharging a liquid medication from a container of the injection device; and
an add-on module comprising a sensor unit, a receiving region adapted to a shape of a device housing of the injection device such that the receiving region is configured to be mounted on the injection device along the longitudinal axis, and a holding mechanism configured to releasably secure the add-on module when mounted to the injection device against moving axially relative to the injection device,
wherein the receiving region is configured such that the discharge button can be contacted when the add-on module is mounted to the injection device, and
wherein the add-on module comprises a release element, which in a release movement for releasing the holding mechanism, can be moved in a plane perpendicular to the longitudinal axis or in a direction at least approximately perpendicular to the longitudinal axis.

2. The injection system according to claim 1, wherein the release element is configured for a linear release movement.

3. The injection system according to claim 1, wherein the release element is provided in a distal half of the add-on module.

4. The injection system according to claim 1, wherein the add-on module comprises a handle, which is configured such that with one hand, a user can actuate the discharge button and at least partially cover the release element when gripping the handle in a first grip position.

5. The injection system according to claim 1, wherein the receiving region is configured to receive the injection device by inserting the injection device in the distal direction, and wherein the injection device comprises a holding surface directed proximally, which engages behind a holding element of the holding mechanism and holds the injection device proximally against a separating movement.

6. The injection system according to claim 5, wherein the injection device comprises a distal carpule holder for receiving the container, wherein the device housing is arranged proximal thereto and configured for receiving a delivery mechanism for discharging the liquid medication from the container, wherein the holding surface is formed by a protrusion or a recess on the carpule holder.

7. The injection system according to claim 5, wherein the release element assumes a stable release position at an end of the release movement.

8. The injection system according to claim 7, wherein by inserting the injection device into the add-on module, the release element executes a holding movement counter to the release movement.

9. The injection system according to claim 8, wherein the holding mechanism comprises a control element, which is moved under tension of a control spring when the injection device is inserted, thereby unblocking the holding movement of the release element.

10. The injection system according to claim 9, wherein the holding movement of the release element is driven by a return spring.

11. The injection system according to claim 5, wherein the injection device comprises at least one of protrusions or recesses, and wherein the add-on module comprises at least one of grooves in a lateral face of the receiving region, which correspond to the protrusions, or holding elements for engagement in the recesses, which correspond to the recesses, and further comprising a further injection device, which differs from the injection device only by the at least one of the protrusions or recesses and a medical indication.

12. The injection system according to claim 1, wherein the add-on module comprises a charging socket for connecting a charging cable configured for charging an energy store of the add-on module, wherein the charging socket is inaccessible when the injection device is inserted into the add-on module.

13. The injection system according to claim 12, wherein the charging socket opens into the receiving region, and wherein the add-on module comprises a lateral opening through which the charging cable can enter and be plugged into the charging socket.

14. The injection system according to claim 1, wherein the add-on module comprises a device cap detector for detecting a device cap when mounted on a carpule holder of the injection device, comprising a tilting element for converting a movement of a first leg end of the tilting element in a direction perpendicular to the longitudinal axis into a movement of a second leg end of the tilting element in an axial direction, and a switching element, which is actuated by an axial movement of the second leg end.

15. The injection system according to claim 1, wherein the add-on module comprises an injection device detector configured detecting when the injection device is inserted into the add-on module, the injection device detector comprising a switching element configured to be actuated by the release element in at least one of a locking movement counter to the release movement or in a subsequently assumed holding position.
